(19) United States
(12) Patent Application Publication
(10) Pub. No.: US 2024/0180095 A1
(43) Pub. Date: Jun. 6, 2024

(54) STAY GREEN CUCURBITACEAE PLANT

(71) Applicant: Enza Zaden Beheer B.V., Enkhuizen (NL)

(72) Inventors: Bart Kaij, Enkhuizen (NL); Yusuf Sen, Enkhuizen (NL); Wilhelmina Antonia Cornelia Anna Leijten, Enkhuizen (NL); Laura Naranjo Peña, Enkhuizen (NL)

(21) Appl. No.: 18/285,048

(22) PCT Filed: Apr. 1, 2021
(86) PCT No.: PCT/EP21/58684

§ 371 Date: Sep. 29, 2023

(51) Int. Cl. A01H 6/34; A01H 1/00; A01H 1/06; A01H 5/08; C07K 14/415

(52) U.S. Cl. CPC A01H 6/34; A01H 1/06; A01H 1/1205; A01H 5/08; C07K 14/415

(57) ABSTRACT

The present invention relates to a Cucurbitaceae plant which provides fruits having an improved shelf life. Further, the present invention relates to seeds, plant tissue, fruits or plants parts of a Cucurbitaceae plant. Further aspect of the present invention relates to a method for providing a Cucurbitaceae plant which provides fruits with an improved shelf life. According to yet another aspect the present invention relates to nucleic acids and amino acids related to the present improved shelf life trait.

The present invention relates to a Cucurbitaceae plant which provides fruits having an improved shelf life. Further, the present invention relates to seeds, plant tissue, fruits or plants parts of a Cucurbitaceae plant. Further aspect of the present invention relates to a method for providing a Cucurbitaceae plant which provides fruits with an improved shelf life. According to yet another aspect the present invention relates to nucleic acids and amino acids related to the present improved shelf life trait.

Cucurbitaceae plants, including squash, pumpkin, cucumber and melon are already cultivated for at least 3.000 years and several different cultivars have emerged, which are grown and market on the global market. The Cucurbitaceae fruits are mainly eaten in the unripe green form since the ripe yellow form normally becomes bitter and sour. Accordingly, the Cucurbitaceae fruits, such as squash and cucumber, are commonly harvested while still green and are generally used for both the pickling industry and the fresh market, which latter usage has the greatest added value for the farmers. Due to its relatively short shelf life, storage and shipping of fresh Cucurbitaceae fruits is, however, difficult and expensive. Cooling, which is generally used to extend the shelf life of fresh products, cannot be applied for Cucurbitaceae fruits as these fruits are not suitable for storage at low temperatures due to chill injury.

It is known to extend the shelf life of Cucurbitaceae fruits by for example wrapping them in sealing foil or storing them under controlled conditions (>10° C.). However, additional processing steps or specific storage measures thus have to be taken. In addition, in this way the shelf life is extended only in the specific Cucurbitaceae that have been wrapped in foil, or have been stored under said specific conditions. Further, although the shelf life is extended by wrapping Cucurbitaceae in foil, these wrapped Cucurbitaceae turn yellow within a time period of about two weeks.

Furthermore, under stress conditions (i.e. drought, disease), Cucurbitaceae plant senescence start very early and leaves become yellow which negatively affects plants health and crop yield. Therefore, it would be of importance to find a solution to inhibit this natural process given the advantage in the field in terms of plant continuation, crop yield and harvest period extension.

Given the above, there is a need in the art for Cucurbitaceae plants providing fruits having an extended shelf life and wherein the process of yellowing of the leaves of the plants is inhibited or reduced.

Therefore, it is an object of the invention, amongst other objects, to provide Cucurbitaceae plants providing fruits having an extend shelf life and wherein the plant and fruits are more resistant to yellowing.

This object, amongst other objects, is met by providing a Cucurbitaceae plant according to the appended claims.

Specifically, this object, amongst other objects, is met by a Cucurbitaceae plant which provides fruits having an improved shelf life, wherein said plant comprises a mutated stay green gene, wherein said stay green gene encodes for a protein having at least 87% sequence identity with SEQ ID No.2 of SEQ ID 6, and wherein the mutated stay green gene comprises at least one mutation in the stay green gene resulting in an amino acid substitution of Serine (S) to Phenylalanine (F) or Leucine (L) at amino acid position 52 or position 54 (S52F, S52L, S54F or S54L), and/or an amino acid substitution of Arginine (R) to Lysine (K) at amino acid position 72 or position 74 (R72K or R74K) in the protein sequence represented by SEQ ID No.2 or SEQ ID No.6, respectively. Preferably the Cucurbitaceae plant of present invention comprises a mutated stay green gene that encodes for a protein comprising the amino acid sequence as shown in SEQ ID NO. 2 or SEQ ID No.6, or amino acid sequences with more than 90% identity, such as more than 91%, 92% or 93%, preferably more than 94% identity such as more than 95%, more preferably more than 96% identity such as more than 97%, even more preferably more than 98% identity or more than 99% identity with SEQ ID NO. 2 or SEQ ID No.6. The expression of said mutated stay green gene is reduced as compared to the expression of a “wild type” stay green gene not comprising the specific mutation, in a Cucurbitaceae plant not providing fruits having an improved shelf life, or the enzymatic activity of said protein is reduced as compared to the enzymatic activity of said protein in a Cucurbitaceae plant not providing fruits having an improved shelf life. The mutated protein comprising the S52F mutation, as shown in SEQ ID NO. 4, or R74K mutation, as shown in SEQ ID NO. 20,ncimb is highly correlated with the improved shelf life trait of the present invention and a stay green phenotype of the plant and its fruit, since an improved shelf life of at least 4 to 5 weeks is observed for fruits from a Cucurbitaceae plant comprising a mutated stay green gene encoding the present protein.

In the present invention, the identified gene encodes for a chloroplast protein and is required for the initiation of chlorophyll breakdown in plants. Modifying this gene function inhibits chlorophyll breakdown and provides a delay in senescence in the field. A mutation was induced using EMS technology providing a mutation in the stay green (SGR) gene in C. pepo and C. melo. It was surprisingly found that an amino acid change at position 52 or 54 from a serine to a phenylalanine or leucine and/or an amino acid change at position 72 or 74 from a arginine to a lysine related to an improved delay of senescence and delay maturity in plant and fruits as compared to other mutations in the stay green gene. Other mutations did not result in the stay green phenotype, such as M1I, A13V, P25S, S30F, G45R, P170L, P188S, P205L, E221K and E251K, as determined in C. pepo and C. melo.

With improved shelf life, as used in the present context, is meant an improved storability of harvested unripe green Cucurbitaceae, which Cucurbitaceae stay green for longer periods of time than comparable standard harvested unripe green Cucurbitaceae. Preferably, the present Cucurbitaceae stay green for a time period up to 4 or 5 weeks. Plants of the invention can be distinguished from prior art plants by measuring the time period under standard storage conditions (about 12° C.) that the Cucurbitaceae fruits stay green. Conventional Cucurbitaceae fruits turn yellow within 1 or 2 weeks; whereas Cucurbitaceae fruits according to the present invention turn yellow after a time period of at least 4 or at least 5 weeks. In addition under stress conditions (i.e. drought, disease), Cucurbitaceae plant senescence is delayed resulting in that leaves maintain their green phenotype for longer periods which positively affects plants health and crop yield. Further. stay green gene expression levels in the present plants and the reference plants can be determined using any suitable and generally known Molecular Biology technique such as a quantitative Polymerase Chain Reaction (PCR) or mRNA hybridization.

According to the present invention, an enzymatic activity is reduced in comparison with the activity of the present protein in a Cucurbitaceae plant which does not provide Cucurbitaceae fruits having an extended shelf life. The term ‘not providing Cucurbitaceae plants having an extended shelf life’ indicates a shelf life, determined in an phenotypic test and appropriate reference plant, such as a parent plant, being less than the shelf life than observed for Cucurbitaceae fruits of the present invention. Suitable reference plants can, besides parent plants, also be plants generally designated as providing marketable Cucurbitaceae fruits.

The present protein has a chlorophyll decarboxylase function wherein, amongst other reactions, a carboxyl group is removed and carbon dioxide is released. Accordingly, the present reduced activity can be determined using an assay measuring compounds being either the starting compounds or the resulting compounds of the enzymatic reaction. As a suitable alternative, protein levels, being inherently indicative of a reduced activity, of the present proteins can be determined by, for example, ELISA or protein hybridization both being techniques commonly known to the skilled person.

The present Cucurbitaceae plants can be obtained by mutagenesis of Cucurbitaceae plants. For example, mutations, either at the expression level or the protein level, can be introduced in these plants by using mutagenic chemicals such as ethyl methane sulfonate (EMS) or by irradiation of plant material with gamma rays or fast neutrons. Mutagenized plants carrying mutations in the present gene can be readily identified by using the TILLING (Targeting Induced Local Lesions IN Genomes) method (McCallum et al. (2000) Targeted screening for induced mutations. Nat. Biotechnol. 18, 455-457, and Henikoff et al. (2004) TILLING. Traditional mutagenesis meets functional genomics. Plant Physiol. 135, 630-636). Briefly, this method is based on the PCR amplification of a gene of interest from genomic DNA of a large collection of mutagenized plants in the M2 generation. By DNA sequencing or by scanning for point mutations using a single-strand specific nuclease, such as the CEL-I nuclease (Till et al. (2004) Mismatch cleavage by single-strand specific nucleases. Nucleic Acids Res. 32, 2632-2641) individual plants having a mutation in the present genes are identified.

According to a preferred embodiment of the present invention the present plants detailed above are not plants exclusively obtained by means of an essentially biological process.

According to a preferred embodiment, the present invention relates to the Cucurbitaceae plant, wherein said plant is one or more selected from the group consisting of Cucurbita pepo (Squash), Cucurbita moschata (Pumpkin), Cucurbita maxima (Pumpkin), Cucurbita argyrosperma (Silver-seed Gourd), Lagenaria siceraria (Bottle gourd), Citrullus lanatus (Watermelon), and Cucumis melo (Melon), preferably Cucurbita pepo and/or Cucumis melo.

According to another preferred embodiment, the present invention relates to the Cucurbitaceae plant, wherein said plant is one or more selected from the group consisting of;
Cucurbita pepo wherein said mutated stay green gene encodes for a protein sequence of SEQ ID No.2 that comprises an amino acid substitution of Serine to Phenylalanine (S52F) at position 52, and/or an amino acid substitution of Arginine (R) to Lysine (K) at position 72 (R72K), corresponding with a nucleotide mutation of C to T at position 155 (C155T) and/or a nucleotide mutation of G to A at position 215 (G215A) in the coding sequence represented by SEQ ID No.1 and/or
Cucumis melo wherein said mutated stay green gene encodes for a protein sequence of SEQ ID No.6 that comprises an amino acid substitution of Serine to Leucine (S54L) at position 54, and/or an amino acid substitution of Arginine (R) to Lysine (K) at position 74 (R74K), corresponding with a nucleotide mutation of C to T at position 161 (C161T) and/or a nucleotide mutation of G to A at position 221 (G221A) in the coding sequence represented by SEQ ID No.5, and/or
Cucurbita moschata wherein said mutated stay green gene encodes for a protein sequence of SEQ ID No.12 that comprises an amino acid substitution of Serine to Phenylalanine (S52F) at position 52, and/or an amino acid substitution of Arginine (R) to Lysine (K) at position 72 (R72K), corresponding with a nucleotide mutation of C to T at position 155 (C155T) and/or a nucleotide mutation of G to A at position 215 (G215A) in the coding sequence represented by SEQ ID No.11, and/or
Cucurbita maxima wherein said mutated stay green gene encodes for a protein sequence of SEQ ID No.10 that comprises an amino acid substitution of Serine to Phenylalanine (S52F) at position 52, and/or an amino acid substitution of Arginine (R) to Lysine (K) at position 72 (R72K), corresponding with a nucleotide mutation of C to T at position 155 (C155T) and/or a nucleotide mutation of G to A at position 215 (G215A) in the coding sequence represented by SEQ ID No.9, and/or
Cucurbita argyrosperma wherein said mutated stay green gene encodes for a protein sequence of SEQ ID No.8 that comprises an amino acid substitution of Serine to Phenylalanine (S52F) at position 52, and/or an amino acid substitution of Arginine (R) to Lysine (K) at position 72 (R72K), corresponding with a nucleotide mutation of C to T at position 155 (C155T) and/or a nucleotide mutation of G to A at position 215 (G215A) in the coding sequence represented by SEQ ID No.7, and/or
Lagenaria siceraria wherein said mutated stay green gene encodes for a protein sequence of SEQ ID No.14 that comprises an amino acid substitution of Serine to Leucine (S52L) at position 52, and/or an amino acid substitution of Arginine (R) to Lysine (K) at position 72 (R72K), corresponding with a nucleotide mutation of C to T at position 155 (C155T) and/or a nucleotide mutation of G to A at position 215 (G215A) in the coding sequence represented by SEQ ID No.13, and/or
Citrullus lanatus wherein said mutated stay green gene encodes for a protein sequence of SEQ ID No.16 that comprises an amino acid substitution of Serine to Leucine (S52L) at position 52, and/or an amino acid substitution of Arginine (R) to Lysine (K) at position 72 (R72K), corresponding with a nucleotide mutation of C to T at position 155 (C155T) and/or a nucleotide mutation of G to A at position 215 (G215A) in the coding sequence represented by SEQ ID No.15, and/or
   The sequence homology among stay green proteins of various Cucurbitaceae plants were analyzed using multiple alignment software and showed that that the SGR proteins share a sequence homology of at least 87%, and preferably at least 90% sequence identity between Cucurbitaceae plants (See also Table 1).

According to a preferred embodiment the present invention relates to the Cucurbitaceae plant, wherein said plant is a C. pepo or C. melo and the mutated stay green gene encodes a protein comprising an amino acid sequence as shown in SEQ ID NO. 4 or SEQ ID NO. 18, respectively.

According to yet a further preferred embodiment, the present stay green gene is present in homozygous form, thereby providing an improved shelf life of the Cucurbitaceae fruits.

According to yet another preferred embodiment, the present invention relates to the Cucurbitaceae plant, wherein said mutated stay green gene is obtainable from deposit number NCIMB 43480 or NCIMB 43513. Seeds of C. pepo and C. melo of present invention are deposited at NCIMB Ltd, Ferguson Building, Craibstone Estate, Bucksburn, Aberdeen AB21 9YA, Scotland, under the number NCIMB 43480 (6 Sep. 2019) and NCIMB 43513 (12 Nov. 2019), respectively.

According to a second aspect, the present invention relates seeds, plant tissue, fruits or plants parts of a Cucurbitaceae plant of present invention comprising a mutated stay green gene providing an improved fruit shelf life. Preferably the stay green gene encodes an amino acid sequence having more than 90% identity, preferably more than 94% identity, more preferably more than 96% identity, even more preferably more than 98% identity with SEQ ID NO. 4 or SEQ ID NO. 18.

According to a further aspect, the present invention relates to a method for providing a Cucurbitaceae plant of present invention, wherein the method comprises the step of providing a Cucurbitaceae plant comprising a mutated stay green gene, wherein said stay green gene encodes for a protein having at least 87% sequence identity with SEQ ID No.2 of SEQ ID 6, and wherein the mutated stay green gene comprises at least one mutation in the stay green gene resulting in an amino acid substitution of Serine (S) to Phenylalanine (F) or Leucine (L) at amino acid position 52 or position 54 (S52F, S52L, S54F or S54L), and/or an amino acid substitution of Arginine (R) to Lysine (K) at amino acid position 72 or position 74 (R72K or R74K) in the protein sequence represented by SEQ ID No.2 or SEQ ID No.6, respectively.

According to a further aspect, the present invention relates to a method for obtaining a Cucurbitaceae plant of present invention which provides fruits having an improved shelf life, wherein the method comprises the steps of,
a) crossing a Cucurbitaceae plant comprised of a mutated stay green gene with a Cucurbitaceae plant that does not comprise said mutated stay green gene,
b) optionally, selfing the plant obtained in step a) for at least one time,
c) selecting the plants that comprise said stay green gene or nucleic acid sequence.

According to a preferred embodiment, the present invention relates to the method for obtaining a Cucurbitaceae plant, wherein the method comprises the step of providing at least one mutation in the stay green gene resulting in an amino acid substitution of Serine (S) to Phenylalanine (F) or Leucine (L) at amino acid position 52 or position 54 (S52F, S52L, S54F or S54L), and/or an amino acid substitution of Arginine (R) to Lysine (K) at amino acid position 72 or position 74 (R72K or R74K) in the protein sequence represented by SEQ ID No.2 or SEQ ID No.6, respectively.

According to another preferred embodiment, the present invention relates to the method for obtaining and providing a Cucurbitaceae plant, wherein the mutation in the stay green gene is obtained by gene editing techniques, preferably by mutagenesis and/or CRISPR/Cas. Preferably the mutation in the stay green gene is a non-natural mutation.

According to a further aspect, the present invention relates to a protein for providing Cucurbitaceae plants which provides fruits with an improved shelf life, wherein the protein comprises SEQ ID No.4 or SEQ ID No.18 or having at least 87% sequence identity with SEQ ID No.4 and SEQ ID No.18. The protein of present invention provides Cucurbitaceae plants that provides fruits with an improved shelf life, or preferably fruits which stay green during storage for a time period of at least 4 or 5 weeks. Furthermore, the protein provides a plant the ability to maintain a green phenotype under stress conditions for a longer period in time, in comparison to plants that do not comprise the mutated stay green gene and protein. Preferably, said mutated stay green protein has more than 90% identity, preferably more than 94% identity, more preferably more than 96% identity, even more preferably more than 98% identity with SEQ ID No.4 and SEQ ID No.18.

According to a further aspect, the present invention relates to a nucleic acid sequence for providing Cucurbitaceae plants which provides fruits with an improved shelf life, wherein the nucleic acid sequence comprises SEQ ID No. 3 or SEQ ID No.17, or having at least 87% sequence identity with SEQ ID No. 3 and SEQ ID No.17. The nucleic acid sequence of present invention provides Cucurbitaceae plants which provides fruits with an improved shelf life, or preferably fruits which stay green during storage for a time period of at least 4 or 5 weeks. Preferably, said nucleic acid has more than 90% identity, preferably more than 94% identity, more preferably more than 96% identity, even more preferably more than 98% identity with SEQ ID NO. 3 and SEQ ID No.17.

According to a further aspect, the present invention relates to use of protein or nucleic acid sequence as indicated above for providing Cucurbitaceae plants which provides fruits with an improved shelf life.

The invention is further elucidated in the illustrative examples below. In the examples, reference is made to figures wherein:

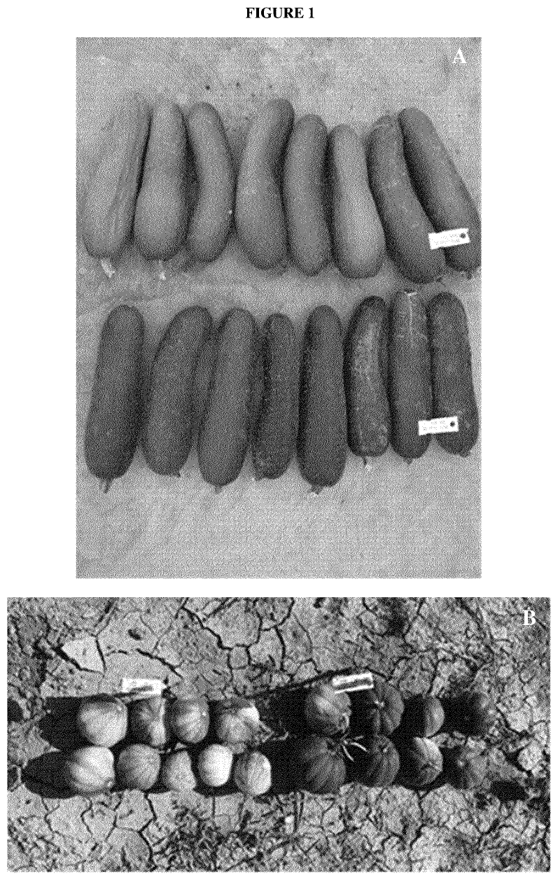

FIG. 1: A) shows a wild type C. pepo fruit (summer squash, zucchini) and C. pepo fruit according to the present invention comprising mutated stay green gene, approximately five weeks after harvest. The wild type C. pepo fruits (upper fruits) become yellow and the peel becomes more wrinkled. The C. pepo of present inventions (lower fruits) remains green and does not show any wrinkling of the skin.
B) shows wild type C. pepo fruit (winter squash, acorn type) and C. pepo fruit according to the present invention, approximately five weeks after harvest. The wild type C. pepo fruits (fruits on the left) become yellow in contrast to the C. pepo of present inventions (fruits on the right) that remain green.

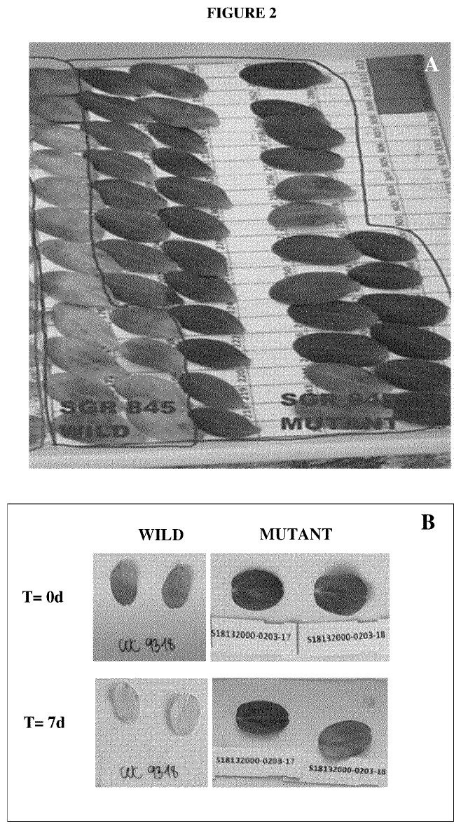

FIG. 2: A) shows leaves of a wild type C. pepo plant (WILD) and a C. pepo plant according to present invention comprising the mutated stay green gene (MUTANT). The leaves of a C. pepo plant of present invention remain green, whereas the wild type plant yellowing of the leaves is observed, after 2 weeks of storage after sampling.
B) shows leaves of a wild type C. melo (WILD) and a C. melo plant according to present invention comprising the mutated stay green gene (MUTANT). The leaves of a C. melo plant of present invention remain green, whereas the wild type plant yellowing of the leaves is observed, after a week (t=7 d) of storage after sampling.

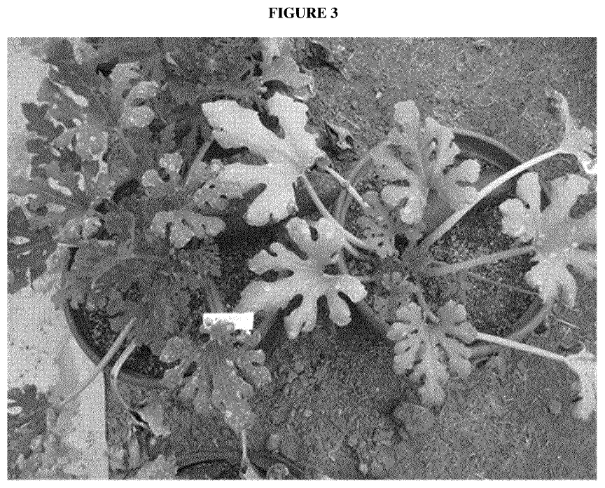

FIG. 3: shows a wild type C. pepo plant (right) and a C. pepo plant according to present invention (left). The plant and leaves of a Cucurbitaceae plant of present invention remain green, whereas at the wild type plant yellowing of the leaves and plant are observed.

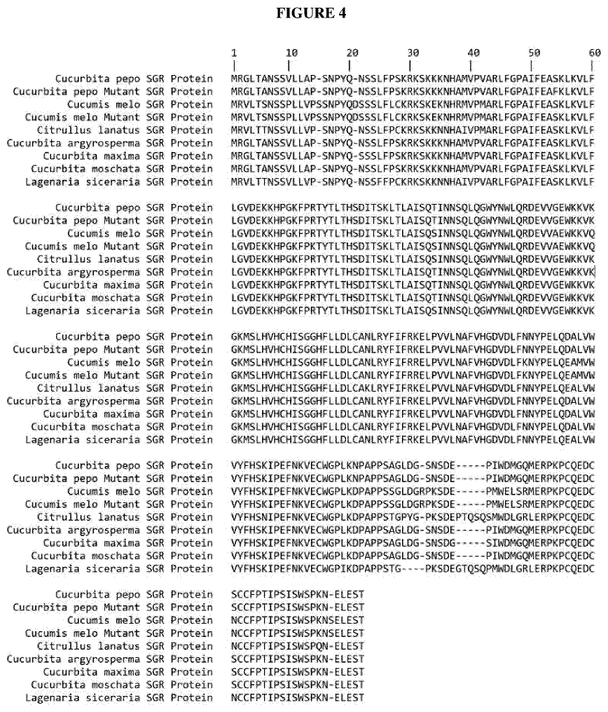

FIG. 4: shows a sequence alignment of the stay green (SGR) protein in various Cucurbitaceae plants; Cucurbita pepo, Cucurbita moschata, Cucurbita maxima, Cucurbita argyrosperma, Lagenaria siceraria, Citrullus lanatus, and Cucumis melo. The alignment also includes the mutated SGR protein in a Cucurbita pepo or Cucumis melo (“Mutant”) according to present invention. The alignments shows that the stay green protein is highly conserved within the Cucurbitaceae family.

### EXAMPLE 1

About two thousand seeds of the C. pepo (breeding line SL0166) were treated with 0.75% (W/V) Ethyl Methane Sulfonate (EMS) for 16 hours. After rinsing the seeds several times with water, seeds were sown in peet blocks. After appearance of the primary leaf, the apical meristem was removed to induce the development of the lateral meristem. This procedure was repeated to induce outgrowth of the lateral meristem of the first side shoot. Samples were taken from the top of this developing shoot for further molecular analysis i.e. the detection of mutations in candidate genes as described by Van Eijk and Van Tunen in EP 1929039. The above EMS procedure was also done for C. melo to produce an EMS mutant population.

Plants derived from the EMS treatment comprising SEQ ID NO. 3 were grown in a greenhouse and fruits where harvested. The shelf life of the fruits was measured during approximately 5 weeks using the following scale: (1) Good colour (green); (2) Acceptable colour (somewhat lighter green); (3) Unacceptable colour (first occurrence of yellowing); (4) Unacceptable colour (yellowing); (5) Unacceptable colour (extreme yellowing). A conventional C. pepo fruit derived from a plant not comprising SEQ ID NO.3 was also measured for approximately 5 weeks.

From the generated EMS population in C. pepo and C. melo, two mutants were selected; the C. pepo (S52F) and C. melo (R74K) mutants outperform the other SGR mutants in terms of maintaining a green colour over a longer period of time, thereby increasing the shelf life of the fruit. Furthermore, several other SGR mutants of C. pepo and C. melo were obtained and analysed for their stay green properties. Next to the S52F and R74K mutant, several other SGR mutants were analysed M1I, A13V, P25S, S30F, G45R, P170L, P188S, P205L, E221K and E251K. Al these other mutants showed yellowing after 2 weeks in storage, comparable to the wild type, non mutated plants.

The C. pepo according to the invention stayed green during the time period of 4 weeks, with a continuous score (1). The conventional C. pepo shows a normal yellowish colour due to chlorophyll degradation during the 5 week test period, reaching score 4 after 5 weeks. FIG. 1A shows a picture with on the top the 5 weeks old conventional zucchini (C. pepo) fruit having a yellowish/orange colour, and below 5 weeks old zucchini according to the present invention having a full green colour. FIG. 1B shows a picture of squash fruit (C. pepo) after 5 weeks of storage, wherein the wild type fruits (fruits on the left) become yellow and the fruits according to present inventions (fruits on the right) remain green.

### EXAMPLE 2

Seeds of plants of example 1 with a mutated stay green gene of SEQ ID No.3 in homozygous form and wild type plants were grown till the cotyledon stage. The cotyledons were removed from the plantlets and placed in a plastic container covered with a glass plate. It can be considered that there was no air exchange possible between the content of the container and the environment. Cotyledons from C. pepo and C. melo plants of seeds that were harvested from the plant with and without the stay green comprising the S52F or R74K mutation, respectively, of present invention in the homozygous form were placed on the grid. After 2 weeks of storage, the picture as shown in FIG. 2 was visible for C. pepo (FIG. 2A) and C. melo (FIG. 2B); the leaves of a C. pepo plant of present invention remain green, whereas the wild type plant yellowing of the leaves is observed.

### EXAMPLE 3

Leaves of a Cucurbitaceae plant, for example C. pepo and C. melo, which are in the lower part of the plant, become old and yellow, start to disintegrate when they are being covered by upper leaves. The disintegration of the leaf is, amongst others, caused by the inactivity and degradation of the chlorophyll. The chlorophyll is giving the green color to the leaf. Plants of the wild type (lacking the present mutant stay green gene), plants with the present stay green gene in heterozygous form, and plants with the present stay green gene comprising the mutation in homozygous form have been planted in the greenhouse. At mature stage of the plants (i.e. after 10 weeks) were placed next to each other. The result is shown in FIG. 3 for C. pepo. It is clear the plant with the present stay green gene in homozygous form is able to maintain its chlorophyll for a longer period. Likewise, it is expected that the plant containing this gene in homozygous form is able to be photosynthetic active for a longer period.

### EXAMPLE 4

The sequence homology among stay green proteins of various Cucurbitaceae plants was analyzed using multiple alignment software. Stay green proteins of Cucurbita pepo, Cucurbita moschata, Cucurbita maxima, Cucurbita argyrosperma, Lagenaria siceraria, Citrullus lanatus, and Cucumis melo were analysed and it was shown that all stay green proteins share a high sequence homology of at least 87%, and in many cases more than 90% sequence identity between Cucurbitaceae plants (Table 1), see also FIG. 4.

TABLE 1
Shows the % sequence homology among stay green (SGR) proteins in Cucurbitaceae plants

| SGR Protein | C. pepo | C. moschata | C. maxima | C. argyro. | L. siceraria | C. lanatus | C. melo |
|---|---|---|---|---|---|---|---|
| C. pepo | — | 99.6 | 98.4 | 99.6 | 89.5 | 88.8 | 87.9 |
| C. moschata | 99.6 | — | 98.8 | 100.0 | 89.9 | 89.1 | 88.3 |
| C. maxima | 98.4 | 98.8 | — | 98.8 | 88.8 | 88.8 | 87.9 |
| C. argyro. | 99.6 | 100.0 | 98.8 | — | 89.9 | 89.1 | 88.3 |
| L. siceraria | 89.5 | 89.9 | 88.8 | 89.9 | — | 95.7 | 87.8 |
| C. lanatus | 88.8 | 89.1 | 88.8 | 89.1 | 95.7 | — | 87.8 |
| C. melo | 87.9 | 88.3 | 87.9 | 88.3 | 87.8 | 87.8 | — |

1. A Cucurbitaceae plant which provides fruits having an improved shelf life, wherein said plant comprises a mutated stay green gene, wherein said stay green gene encodes for a protein having at least 87% sequence identity with SEQ ID No.2 of SEQ ID 6, and wherein the mutated stay green gene comprises at least one mutation in the stay green gene resulting in an amino acid substitution of Serine (S) to Phenylalanine (F) or Leucine (L) at amino acid position 52 or position 54 (S52F, S52L, S54F or S54L), and/or an amino acid substitution of Arginine (R) to Lysine (K) at amino acid position 72 or position 74 (R72K or R74K) in the protein sequence represented by SEQ ID No.2 or SEQ ID No.6, respectively.

2. Cucurbitaceae plant according to claim 1, wherein said plant is one or more selected from the group consisting of Cucurbita pepo, Cucurbita moschata, Cucurbita maxima, Cucurbita argyrosperma, Lagenaria siceraria, Citrullus lanatus, and Cucumis melo, preferably Cucurbita pepo and/or Cucumis melo.

3. Cucurbitaceae plant according to claim 1 or 2, wherein said plant is;
Cucurbita pepo wherein said mutated stay green gene encodes for a protein sequence of SEQ ID No.2 that comprises an amino acid substitution of Serine to Phenylalanine (S52F) at position 52, and/or an amino acid substitution of Arginine (R) to Lysine (K) at position 72 (R72K), and/or
Cucumis melo wherein said mutated stay green gene encodes for a protein sequence of SEQ ID No.6 that comprises an amino acid substitution of Serine to Leucine (S54L) at position 54, and/or an amino acid substitution of Arginine (R) to Lysine (K) at position 74 (R74K), and/or
Cucurbita moschata wherein said mutated stay green gene encodes for a protein sequence of SEQ ID No.12 that comprises an amino acid substitution of Serine to Phenylalanine (S52F) at position 52, and/or an amino acid substitution of Arginine (R) to Lysine (K) at position 72 (R72K), and/or
Cucurbita maxima wherein said mutated stay green gene encodes for a protein sequence of SEQ ID No.10 that comprises an amino acid substitution of Serine to Phenylalanine (S52F) at position 52, and/or an amino acid substitution of Arginine (R) to Lysine (K) at position 72 (R72K), and/or
Cucurbita argyrosperma wherein said mutated stay green gene encodes for a protein sequence of SEQ ID No.8 that comprises an amino acid substitution of Serine to Phenylalanine (S52F) at position 52, and/or an amino acid substitution of Arginine (R) to Lysine (K) at position 72 (R72K), and/or
Lagenaria siceraria wherein said mutated stay green gene encodes for a protein sequence of SEQ ID No.14 that comprises an amino acid substitution of Serine to Leucine (S52L) at position 52, and/or an amino acid substitution of Arginine (R) to Lysine (K) at position 72 (R72K), and/or
Citrullus lanatus wherein said mutated stay green gene encodes for a protein sequence of SEQ ID No.16 that comprises an amino acid substitution of Serine to Leucine (S52L) at position 52, and/or an amino acid substitution of Arginine (R) to Lysine (K) at position 72 (R72K).

4. Cucurbitaceae plant according to any one of the claims 1 to 3, wherein said plant is a C. pepo or C. melo and the mutated stay green gene encodes a protein comprising an amino acid sequence as shown in SEQ ID NO. 4 or SEQ ID NO. 18, respectively.

5. Cucurbitaceae plant according to any one of the claims 1 to 4, wherein said stay green gene is present in homozygous form.

6. Cucurbitaceae plant according to any one of the claims 1 to 5, wherein said mutated stay green gene is obtainable from deposit number NCIMB 43480 or NCIMB 43513.

7. Seeds, plant tissue, fruits or plants parts of a Cucurbitaceae plant according to any one of the claims 1 to 6 comprising a mutated stay green gene providing an improved fruit shelf life.

8. Method for providing a Cucurbitaceae plant according to any one of the claims 1 to 6, wherein the method comprises the step of providing a Cucurbitaceae plant comprising a mutated stay green gene, wherein said stay green gene encodes for a protein having at least 87% sequence identity with SEQ ID No.2 of SEQ ID 6, and wherein the mutated stay green gene comprises at least one mutation in the stay green gene resulting in an amino acid substitution of Serine (S) to Phenylalanine (F) or Leucine (L) at amino acid position 52 or position 54 (S52F, S52L, S54F or S54L), and/or an amino acid substitution of Arginine (R) to Lysine (K) at amino acid position 72 or position 74 (R72K or R74K) in the protein sequence represented by SEQ ID No.2 or SEQ ID No.6, respectively.

9. Method for obtaining a Cucurbitaceae plant of any one of the claims 1 to 6 which provides fruits having an improved shelf life, wherein the method comprises the steps of,
a) crossing a Cucurbitaceae plant comprised of a mutated stay green gene with a Cucurbitaceae plant that does not comprise said mutated stay green gene,
b) optionally, selfing the plant obtained in step a) for at least one time,
c) selecting the plants that comprise said stay green gene or nucleic acid sequence.

10. Method for obtaining a Cucurbitaceae plant according to claim 9, wherein the method comprises the step of providing at least one mutation in the stay green gene resulting in an amino acid substitution of Serine (S) to Phenylalanine (F) or Leucine (L) at amino acid position 52 or position 54 (S52F, S52L, S54F or S54L), and/or an amino acid substitution of Arginine (R) to Lysine (K) at amino acid position 72 or position 74 (R72K or R74K) in the protein sequence represented by SEQ ID No.2 or SEQ ID No.6, respectively.

11. Method according to claims 8 to 10, wherein the mutation in the stay green gene is obtained by gene editing techniques, preferably by mutagenesis and/or CRISPR/Cas.

12. Method according to any one of the claims 8 to 11, wherein the mutation in the stay green gene is a non-natural mutation.

13. Protein for providing Cucurbitaceae plants which provides fruits with an improved shelf life, wherein the protein comprises SEQ ID No.4 or SEQ ID No.18 or having at least 87% sequence identity with SEQ ID No.4 and SEQ ID No.18.

14. Nucleic acid sequence for providing Cucurbitaceae plants which provides fruits with an improved shelf life, wherein the nucleic acid sequence comprises SEQ ID No. 3 or SEQ ID No.17, or having at least 87% sequence identity with SEQ ID No. 3 and SEQ ID No.17.

15. Use of protein or nucleic acid sequence according to claim 13 or 14 for providing Cucurbitaceae plants which provides fruits with an improved shelf life.


SEQUENCE LISTING (18 sequences)

SEQ ID NO: 1 - DNA, 762 bp, Cucurbita pepo
  >Cucurbita_pepo_SGR_CDS
atgaggggtttgacggcgaattcttctgttcttcttgctccttcaaacccatatcagaattcctctctgtttccttctaaacgcaagtccaagaagaagaaccatgccatggttcctgtagcgaggctgtttgggccggccatatttgaagcttccaagttgaaggttctgtttttaggagtggatgagaagaagcatcctggaaaatttcccaggacttacacgcttacacatagcgatattacctctaaacttactctcgctatttctcagaccattaacaactctcagctgcaaggatggtataattggctacagagagatgaagtggtgggagagtggaagaaggtgaagggaaagatgtcgcttcatgttcattgccatatcagcggcggccattttcttttagatctctgtgctaatctcagatacttcatcttccgcaaagaactccctgtggttctgaatgcatttgtccatggagatgtggacttgttcaacaattacccagaattacaggacgcgttggtctgggtttatttccattcgaagatcccagaattcaacaaagttgaatgctggggcccactcaagaatccagccccaccttcagctggactggatgggtccaattcagatgagcccatatgggatatgggccaaatggagcggcccaaaccttgccaagaagactgctcttgttgcttcccaaccatcccttccatttcatggtcacccaaaaatgagttggagagtacctga

SEQ ID NO: 2 - protein, 253 aa, Cucurbita pepo
  >Cucurbita_pepo_SGR_Protein
MRGLTANSSVLLAPSNPYQNSSLFPSKRKSKKKNHAMVPVARLFGPAIFEASKLKVLFLGVDEKKHPGKFPRTYTLTHSDITSKLTLAISQTINNSQLQGWYNWLQRDEVVGEWKKVKGKMSLHVHCHISGGHFLLDLCANLRYFIFRKELPVVLNAFVHGDVDLFNNYPELQDALVWVYFHSKIPEFNKVECWGPLKNPAPPSAGLDGSNSDEPIWDMGQMERPKPCQEDCSCCFPTIPSISWSPKNELEST

SEQ ID NO: 3 - DNA, 762 bp, Cucurbita pepo
  >Cucurbita_pepo_Mutant_SGR_CDS
atgaggggtttgacggcgaattcttctgttcttcttgctccttcaaacccatatcagaattcctctctgtttccttctaaacgcaagtccaagaagaagaaccatgccatggttcctgtagcgaggctgtttgggccggccatatttgaagctttcaagttgaaggttctgtttttaggagtggatgagaagaagcatcctggaaaatttcccaggacttacacgcttacacatagcgatattacctctaaacttactctcgctatttctcagaccattaacaactctcagctgcaaggatggtataattggctacagagagatgaagtggtgggagagtggaagaaggtgaagggaaagatgtcgcttcatgttcattgccatatcagcggcggccattttcttttagatctctgtgctaatctcagatacttcatcttccgcaaagaactccctgtggttctgaatgcatttgtccatggagatgtggacttgttcaacaattacccagaattacaggacgcgttggtctgggtttatttccattcgaagatcccagaattcaacaaagttgaatgctggggcccactcaagaatccagccccaccttcagctggactggatgggtccaattcagatgagcccatatgggatatgggccaaatggagcggcccaaaccttgccaagaagactgctcttgttgcttcccaaccatcccttccatttcatggtcacccaaaaatgagttggagagtacctga

SEQ ID NO: 4 - protein, 253 aa, Cucurbita pepo
  >Cucurbita_pepo_Mutant_SGR_Protein
MRGLTANSSVLLAPSNPYQNSSLFPSKRKSKKKNHAMVPVARLFGPAIFEAFKLKVLFLGVDEKKHPGKFPRTYTLTHSDITSKLTLAISQTINNSQLQGWYNWLQRDEVVGEWKKVKGKMSLHVHCHISGGHFLLDLCANLRYFIFRKELPVVLNAFVHGDVDLFNNYPELQDALVWVYFHSKIPEFNKVECWGPLKNPAPPSAGLDGSNSDEPIWDMGQMERPKPCQEDCSCCFPTIPSISWSPKNELEST

SEQ ID NO: 5 - DNA, 774 bp, Cucumis melo
  >Cucumis_melo_SGR_CDS
atgagggttttgactagtaattcttctcctcttcttgttccttcttcaaacccttatcaggattcttcttctctcttcctctgtaaacgcaaatccaaggagaaaaaccacagaatggttcctatggcgagattgtttgggccagccatatttgaagcttcgaagctaaaggttctgtttttaggggtggatgagaagaaacatccaggcaaatttccaaggacttatacgcttacacatagtgatattacttctaaacttactcttgccatttctcaatccattaacaattctcagttacaaggatggtataattggcttcaaagggatgaagtggtagcagaatggaagaaagtacaggggaaaatgtcccttcatgttcattgtcatatcagtggtggccattttcttctagatctctgtgctaatctccgatacttcatctttcgcagagaacttcctgtggtgctgaatgctttcgtccatggagatgtggacttgttcaagaattacccagagctacaagaggctatggtttgggtttatttccactccaaaattccggaattcaacaaagtagaatgctggggcccactaaaggatccagccccaccttcaagtgggcttgatgggcggcccaaatcagatgagcccatgtgggaattgagccggatggagcggcccaaaccttgccaagaagactgcaattgttgcttcccaaccatcccttccatttcatggtcccccaagaacagtgagttggagagcacgtga

SEQ ID NO: 6 - protein, 257 aa, Cucumis melo
  >Cucumis_melo_SGR_Protein
MRVLTSNSSPLLVPSSNPYQDSSSLFLCKRKSKEKNHRMVPMARLFGPAIFEASKLKVLFLGVDEKKHPGKFPRTYTLTHSDITSKLTLAISQSINNSQLQGWYNWLQRDEVVAEWKKVQGKMSLHVHCHISGGHFLLDLCANLRYFIFRRELPVVLNAFVHGDVDLFKNYPELQEAMVWVYFHSKIPEFNKVECWGPLKDPAPPSSGLDGRPKSDEPMWELSRMERPKPCQEDCNCCFPTIPSISWSPKNSELEST

SEQ ID NO: 7 - DNA, 762 bp, Cucurbita argyrosperma
  >Cucurbita_argyrosperma_SGR_CDS
atgaggggtttgacggcgaattcttctgttcttcttgctccttcaaacccatatcagaattcctctctgtttccttctaaacgcaagtccaagaagaagaaccatgccatggttcctgtagcgaggctgtttgggccggccatatttgaagcttccaagttaaaggttctgtttttaggagtggatgaaaagaagcatcctggcaaatttcccaggacttacacgcttacacatagcgatattacctctaaacttactctcgctatttctcagaccattaacaactctcagctgcaaggatggtataattggctacaaagagatgaagtggtgggagagtggaagaaggtgaagggaaagatgtcgcttcatgttcattgccatatcagcggcggccattttcttttagatctctgtgctaatctcagatacttcatcttccgcaaagaactccctgtggttctgaatgcgtttgtccatggagatgtggacttgttcaacaattacccagaattacaggacgcgttggtctgggtttatttccattcgaagatcccagaattcaacaaagttgaatgctggggcccactcaaggatccagccccaccttcagctggacttgatgggtccaattcagatgagcccatatgggatatgggccaaatggagcggcccaaaccttgccaagaagactgctcttgttgcttcccaaccatcccttccatttcatggtcacccaaaaatgagttggagagtacctga

SEQ ID NO: 8 - protein, 253 aa, Cucurbita argyrosperma
  >Cucurbita_argyrosperma_SGR_Protein
MRGLTANSSVLLAPSNPYQNSSLFPSKRKSKKKNHAMVPVARLFGPAIFEASKLKVLFLGVDEKKHPGKFPRTYTLTHSDITSKLTLAISQTINNSQLQGWYNWLQRDEVVGEWKKVKGKMSLHVHCHISGGHFLLDLCANLRYFIFRKELPVVLNAFVHGDVDLFNNYPELQDALVWVYFHSKIPEFNKVECWGPLKDPAPPSAGLDGSNSDEPIWDMGQMERPKPCQEDCSCCFPTIPSISWSPKNELEST

SEQ ID NO: 9 - DNA, 762 bp, Cucurbita maxima
  >Cucurbita_maxima_SGR_CDS
atgaggggtttgacggcgaattcttctgttcttcttgctccttcaaacccatatcagagttcctctctgtttccttctaaacgcaagtccaagaagaagaaccatgctatggttcctgtagcgaggctgtttgggccggccatatttgaagcttccaagttgaaggttctgtttttaggagtggatgagaagaagcatcctggaaaatttcccaggacttacacgcttacacacagcgatattacctctaaacttactctcgctatttctcagaccattaacaactctcagctgcaaggatggtataattggctacaaagagatgaagtggtgggagagtggaagaaggtgaagggaaagatgtcgcttcatgttcattgccatatcagcggcggccattttcttttagatctctgtgctaatctcagatacttcatcttccgcaaagaactccctgtggttctgaatgcgtttgtccatggagatgtggatttattcaacaattacccagaattacaggatgcgttggtctgggtttatttccattcaaagatcccagaattcaacaaagttgaatgctggggcccactcaaggatccagccccaccttcagctggacttgatgggtccaattcagacgggtccatatgggatatgggccaaatggagcggcccaaaccttgccaagaagactgctcttgttgcttcccaaccatcccttccatttcatggtcacccaaaaatgagttggagagtacctga

SEQ ID NO: 10 - protein, 253 aa, Cucurbita maxima
  >Cucurbita_maxima_SGR_Protein
MRGLTANSSVLLAPSNPYQSSSLFPSKRKSKKKNHAMVPVARLFGPAIFEASKLKVLFLGVDEKKHPGKFPRTYTLTHSDITSKLTLAISQTINNSQLQGWYNWLQRDEVVGEWKKVKGKMSLHVHCHISGGHFLLDLCANLRYFIFRKELPVVLNAFVHGDVDLFNNYPELQDALVWVYFHSKIPEFNKVECWGPLKDPAPPSAGLDGSNSDGSIWDMGQMERPKPCQEDCSCCFPTIPSISWSPKNELEST

SEQ ID NO: 11 - DNA, 762 bp, Cucurbita moschata; identical to SEQ ID NO: 7
  >Cucurbita_moschata_SGR_CDS

SEQ ID NO: 12 - protein, 253 aa, Cucurbita moschata; identical to SEQ ID NO: 8
  >Cucurbita_moschata_SGR_Protein

SEQ ID NO: 13 - DNA, 768 bp, Lagenaria siceraria
  >Lagenaria_siceraria_SGR_CDS
atgagggttttgacgactaattcttctgttcttcttgttccttcaaacccatatcagaattcttctttcttcccctgtaaacgcaaatccaagaagaacaaccatgccatagtccctgtggcgagactatttgggccagctatatttgaagcttcgaagctgaaggttctgtttttaggagtggatgagaagaagcatccagggaaatttccaaggacttatacgcttacacatagcgatataacttctaaactcactcttgccatttctcagtccattaacaactctcagttacaaggatggtataattggctgcaaagggatgaagtggtgggagaatggaagaaagtgaagggaaaaatgtctcttcatgttcattgccatattagtggtggccattttcttttagatctttgtgctaatctcagatacttcatctttcgcaaagaacttcctgtggtgctgaatgcttttgtgcatggagatgtggacttgttcaacaattacccagaattacaggaggctttggtttgggtttatttccactccaaaattccggaattcaataaagtagaatgttggggcccaataaaggatccagccccaccttcaactgggcccaaatcagatgagggcactcaaagccagcccatgtgggatttgggccggctggagcggcccaaaccttgccaagaagactgcaattgttgcttcccaaccatcccttccatttcatggtcccccaaaaatgagttggagagcacctga

SEQ ID NO: 14 - protein, 255 aa, Lagenaria siceraria
  >Lagenaria_siceraria_SGR_Protein
MRVLTTNSSVLLVPSNPYQNSSFFPCKRKSKKNNHAIVPVARLFGPAIFEASKLKVLFLGVDEKKHPGKFPRTYTLTHSDITSKLTLAISQSINNSQLQGWYNWLQRDEVVGEWKKVKGKMSLHVHCHISGGHFLLDLCANLRYFIFRKELPVVLNAFVHGDVDLFNNYPELQEALVWVYFHSKIPEFNKVECWGPIKDPAPPSTGPKSDEGTQSQPMWDLGRLERPKPCQEDCNCCFPTIPSISWSPKNELEST

SEQ ID NO: 15 - DNA, 777 bp, Citrullus lanatus
  >Citrullus_lanatus_SGR_CDS
atgagggttttgacgactaattcttctgttcttcttgttccttcaaacccatatcagaattcttctctcttcccctgtaaacgcaaatccaagaagaacaaccatgccatagtccctatggccaggctatttgggccagctatatttgaagcttcgaagctgaaggttctgtttttaggagtggatgagaagaagcatccaggaaaatttccaagaacttatacgctaactcatagcgatataacttctaaactcactcttgccatttctcagtccattaacaactctcagttacaaggatggtacaattggctgcaaagggatgaagtggtaggagaatggaagaaagtgaaaggaaaaatgtctcttcatgttcattgccatattagtggtggccattttcttttagatctttgtgctaaactcagatacttcatctttcgcaaagaacttcctgtggtgctgaatgcttttgtccatggagatgtggacttgttcaacaattacccagaattacaagaggctttggtttgggtttattttcactccaatattccagaattcaacaaagtggaatgttggggcccattaaaggatccagccccaccttcaactgggccctatgggcccaagtcagatgagcccactcaaagccagtccatgtgggatttgggccggctggagcggcccaaaccttgccaagaagattgcaattgttgcttcccaaccatcccttccatttcatggtccccccaaaatgagttggagagcacctga

SEQ ID NO: 16 - protein, 258 aa, Citrullus lanatus
  >Citrullus_lanatus_SGR_Protein
MRVLTTNSSVLLVPSNPYQNSSLFPCKRKSKKNNHAIVPMARLFGPAIFEASKLKVLFLGVDEKKHPGKFPRTYTLTHSDITSKLTLAISQSINNSQLQGWYNWLQRDEVVGEWKKVKGKMSLHVHCHISGGHFLLDLCAKLRYFIFRKELPVVLNAFVHGDVDLFNNYPELQEALVWVYFHSNIPEFNKVECWGPLKDPAPPSTGPYGPKSDEPTQSQSMWDLGRLERPKPCQEDCNCCFPTIPSISWSPQNELEST

SEQ ID NO: 17 - DNA, 774 bp, Cucumis melo
  cDNA SGR mutant Cucumis Melo
atgagggttttgactagtaattcttctcctcttcttgttccttcttcaaacccttatcaggattcttcttctctcttcctctgtaaacgcaaatccaaggagaaaaaccacagaatggttcctatggcgagattgtttgggccagccatatttgaagcttcgaagctaaaggttctgtttttaggggtggatgagaagaaacatccaggcaaatttccaaagacttatacgcttacacatagtgatattacttctaaacttactcttgccatttctcaatccattaacaattctcagttacaaggatggtataattggcttcaaagggatgaagtggtagcagaatggaagaaagtacaggggaaaatgtcccttcatgttcattgtcatatcagtggtggccattttcttctagatctctgtgctaatctccgatacttcatctttcgcagagaacttcctgtggtgctgaatgctttcgtccatggagatgtggacttgttcaagaattacccagagctacaagaggctatggtttgggtttatttccactccaaaattccggaattcaacaaagtagaatgctggggcccactaaaggatccagccccaccttcaagtgggcttgatgggcggcccaaatcagatgagcccatgtgggaattgagccggatggagcggcccaaaccttgccaagaagactgcaattgttgcttcccaaccatcccttccatttcatggtcccccaagaacagtgagttggagagcacgtga

SEQ ID NO: 18 - protein, 257 aa, Cucumis melo
  protein SGR mutant Cucumis Melo
MRVLTSNSSPLLVPSSNPYQDSSSLFLCKRKSKEKNHRMVPMARLFGPAIFEASKLKVLFLGVDEKKHPGKFPKTYTLTHSDITSKLTLAISQSINNSQLQGWYNWLQRDEVVAEWKKVQGKMSLHVHCHISGGHFLLDLCANLRYFIFRRELPVVLNAFVHGDVDLFKNYPELQEAMVWVYFHSKIPEFNKVECWGPLKDPAPPSSGLDGRPKSDEPMWELSRMERPKPCQEDCNCCFPTIPSISWSPKNSELEST